(19) United States
(12) Patent Application Publication
(10) Pub. No.: US 2017/0042507 A1
(43) Pub. Date: Feb. 16, 2017

(54) HIGH VOLTAGE MEMS, AND A PORTABLE ULTRASOUND DEVICE COMPRISING SUCH A MEMS

(71) Applicant: NovioScan B.V., Nijmegen (NL)

(72) Inventors: Jan Jacob Koning, Nijmegen (NL); Reinout Woltjer, Nijmegen (NL)

(21) Appl. No.: 15/261,483

(22) Filed: Sep. 9, 2016

Related U.S. Application Data
(63) Continuation of application No. PCT/NL2015/050160, filed on Mar. 13, 2015.

(30) Foreign Application Priority Data
Mar. 13, 2014 (NL) 2012419

(51) Int. Cl. A61B 8/00; H01L 41/083; H01L 41/113; A61F 5/48; H01L 41/047; H01L 41/04; A61B 5/20; A61B 8/08; H01L 41/09; H01L 41/187

(52) U.S. Cl. CPC A61B 8/4427; H01L 41/094; H01L 41/083; H01L 41/0933; H01L 41/0973; H01L 41/1132; H01L 41/1876; H01L 41/1873; H01L 41/0471; H01L 41/042; A61B 5/204; A61B 8/08; A61B 8/4494; A61B 8/5207; A61F 5/48; A61B 2562/028

(57) ABSTRACT

An improved high voltage MEMS, and a portable ultrasound device comprising such a MEMS, and use of such a portable device for detecting a liquid volume. Microelectromechanical systems (MEMS) relate to a technology of very small devices. Piezoelectricity relates at one hand to accumulation of electric charge in certain solid materials in response to an applied mechanical stress.

### CROSS-REFERENCE TO RELATED APPLICATIONS

This application is a continuation application of Patent Cooperation Treaty Application No. PCT/NL2015/050160, filed on Mar. 13, 2015, which claims priority to Netherlands Patent Application No. 2012419, filed on Mar. 13, 2014, and the specifications and claims thereof are incorporated herein by reference.

### STATEMENT REGARDING FEDERALLY SPONSORED RESEARCH OR DEVELOPMENT

Not Applicable.

### THE NAMES OF PARTIES TO A JOINT RESEARCH AGREEMENT

Not Applicable.

### INCORPORATION BY REFERENCE OF MATERIAL SUBMITTED ON A COMPACT DISC

Not Applicable.

### STATEMENT REGARDING PRIOR DISCLOSURES BY THE INVENTOR OR A JOINT INVENTOR

Not Applicable.

### COPYRIGHTED MATERIAL

Not Applicable.

### BACKGROUND OF THE INVENTION

Field of the Invention (Technical Field)

The present invention relates to the field of improved high voltage MEMS, and a portable ultrasound device comprising such a MEMS, and use of such a portable device for detecting a liquid volume.

Description of Related Art Including Information Disclosed Under 37 C.F.R. §§1.97 and 1.98

Many people like elderly persons, women after delivery of a baby, lesion patients, dement people, and others, have a difficulty to control functioning of the bladder, and to be at the toilet on time to urinate. This (partial) incontinency is highly inconvenient and may also be an item that is not addressed at all. Solutions to this incontinency relate to applications of e.g. diapers. However, such is effectively not more than limiting consequences of incontinency, without providing a real solution, and still a bad odor, wet pants/dresses cannot be prevented. Such solutions also cost a considerable amount of money.

Ultrasound is an oscillating sound pressure wave with a frequency greater than the upper limit of the human hearing range (hence ultra-sound). Ultrasound devices may operate with frequencies from 20 kHz up to several gigahertzes. Ultrasound may be used in many different fields. Ultrasonic devices are used to detect objects and measure distances. Ultrasonic imaging (sonography) is used in both veterinary medicine and human medicine. In the nondestructive testing of products and structures, ultrasound is used to detect invisible flaws. Industrially, ultrasound is used for cleaning and for mixing, and to accelerate chemical processes. Ultrasonics relates to application of ultrasound. Ultrasound can be used for medical imaging, detection, measurement and cleaning. At higher power levels, ultrasonics may be useful for changing the chemical properties of substances.

For some ultrasound applications capacitive (MEMS-) based devices are used. These capacitive based devices suffer from various drawbacks, amongst others a bad reliability.

Ultrasound devices are typically large, or at least too large to be carried, require thick cabling in view of high voltages needed, and are not practical for monitoring.

In principle ultrasound could be used to monitor and measure an amount of fluid, such as being present in a human body. However, monitoring over longer times is typically not possible with prior art devices that have a handheld transducer.

Incidentally various documents recite ultrasound applications.

US2013294201 (A1) recites wide bandwidth piezoelectric micromachined ultrasonic transducers (pMUTs), pMUT arrays and systems having wide bandwidth pMUT arrays are described herein. For example, a piezoelectric micromachined ultrasonic transducer (pMUT) includes a piezoelectric membrane disposed on a substrate. A reference electrode is coupled to the membrane. First and second drive/sense electrodes are coupled to the membrane to drive or sense a first and second mode of vibration in the mem-brane.

This document relates to horizontal arrays of mem-branes, mostly being circular symmetric. The membranes are not stacked. The membranes are excited by two coplanar electrodes on one side of the piezoelectric layer.

WO2009153757 (A1) recites a piezoelectric bimorph switch, specifically a cantilever (single clamped beam) switch, which can be actively opened and closed. The switch thereof comprises piezoelectric stack layers, which form a symmetrical stack, wherein an electric field is always ap-plied in the same direction as the poling direction of the piezoelectric layers.

Therefore the piezoelectric layers are poled to-wards one and another, with one central electrode, and further a bottom and a top electrode; by nature the poling of piezoelectric layers is therefore in opposite direction and no application for ultrasound is mentioned.

US2013023786 (A1) recites an apparatus comprising a sensing unit configured to predict a release of urine using one or more prediction parameters, detect an actual re-lease of urine and adapt at least one of the one or more prediction parameters based on the actual release of urine to increase prediction accuracy.

This document is silent on the construction of transducers and the presence of a MEMS.

US2009069688 (A1) recites an ultrasound probe including a first substrate having a silicon substrate and an ultrasound transmit-receive element, an acoustic lens disposed over an upper surface of the first substrate, and a damping layer disposed under the first substrate, in which a second substrate is disposed between a lower surface of the first substrate and an upper surface of the damping layer, and the second substrate is made of a material having approximately the same linear expansion coefficient and acoustic impedance as the silicon substrate of the first substrate. With this structure, it is possible to provide the ultrasound probe which can prevent damage to the silicon substrate due to temperature change and has excellent transmission/reception performance and structure reliability while reducing noise by reflected waves in transmission and reception.

The detection of ultrasound makes use of a capacitor, and the gap between two electrodes and no piezoelectric material is implemented.

US2010192842 (A1) recites a film formed on a substrate different from the monocrystalline perovskite substrate that contains a piezoelectric oxide.

Apparently it relates to replacing lead (Pb) with Bi in perovskite single layers. Otherwise the document is irrelevant for the present application.

U.S. Pat. No. 6,114,797 (A) recites circuits for use in ignition systems which use a resonating piezoelectric transformer along with complementary circuit components, to efficiently convert a DC first voltage to a transformer-output AC second voltage. The transformer circuit may be a “self-resonating” circuit. In a modified circuit the circuit is not “self-resonating” and instead has a phase shift oscillator sub-circuit that provides small pulse signals to start the transformer resonating when the circuit is initially turned on.

The self-resonating aspect and especially pro-longed time needed therefore is irrelevant for the present invention. Further other aspects, such as bonding of layers, and construction of electrodes, make the disclosure practically incompatible with the present invention.

Therefore there still is a need for an improved MEMS, and likewise for an ultrasound device, which overcome one or more of the above disadvantages, without jeopardizing functionality and advantages.

### BRIEF SUMMARY OF THE INVENTION

The present invention relates in a first aspect to a high voltage MEMS according to claim 10, in a second aspect to a portable ultrasound transducer according to claim 1, in a third aspect to a method of operating an ultrasound device according to claim 9, in a fourth aspect to a method of operating a MEMS according to claim 14, and in a fifth aspect to a membrane according to claim 15.

The high voltage of the present MEMS in operation is typically 20-500 V, preferably 50-250 V, such as 80-100 V. It is noted that especially for small devices, such as devices comprising semiconductor material, such as a chip containing integrated circuit components, such a voltage is considered “high”. The present MEMS requires a high volt-age. Such MEMS have not been available, e.g. due to a break down voltage of below 50 V in typical semiconductor processes. Further prior art piezoelectric based MEMS still suffer from many problems, such as blister forming, break-down at low voltage, non-uniformity of layer thickness over the silicon wafer, loss of internal polarization field (if present) at limited temperatures of some 300° C., a need to polarize the internal field after layer deposition, bumpy frequency characteristics, too much energy consumption, or brittle layers which are hard to dice into separate devices. The present MEMS provides excellent layer uniformity (of better than 2% over a wafer), an internal polarization field in direction of growth which is built in at growth temperature of 450° C. to 600° C. good eigen-frequency properties, well defined frequency characteristics, good method of definition by lithographic chip manufacturing techniques with high reproducibility and low variation from device to device, and ability for wafer separation into chips.

The present MEMS comprises a stack of layers having at least two piezoelectric elements poled in a same direction, i.e. “upwards” or “downwards”. The at least two piezoelectric elements may be stacked horizontally, vertically, and combinations thereof. In an example the present MEMS relates to a cantilever. Therewith the present MEMS, and the present ultrasound device, can be operated at a relatively high voltage for a device comprising a MEMS, such as at a voltage of 80 V. Such is typically not possible with a prior art MEMS. In principle each piezoelectric element has a similar or the same voltage size applied thereon, such as 40 V. A first piezoelectric element may have a negative voltage, such as −50V, and a second piezo-electric element may have a positive voltage, such as +50V. The piezoelectric elements may have a shared central electrode (see e.g. FIG. 1a).

The present piezoelectric elements are provided with a top and bottom electrode, respectively, for providing an electrical field. Therewith the piezoelectric layer will change in size, that is, increase or decrease in size. If the layer is (partly) confined, increase (elongation) and decrease (shrinkage) will result in bending of the MEMS. IF an alternating electrical field is applied, the MEMS will follow the field by bending “upwards” and “downwards”, respectively. Therewith the MEMS can oscillate with a certain frequency, the frequency being applied by e.g. the electrical field.

In between the piezoelectric elements at least one dielectric layer may be provided.

The top electrode covers the piezoelectric layer completely or partially, i.e. covering 1-100% of the piezoelectric layer, e.g. 5-90%, 10-80%, 20-70%, such as 50-60%. Likewise the piezoelectric layer covers the bottom electrode completely or partially, i.e. covering 1-100% of the piezoelectric layer, e.g. 5-90%, 10-80%, 20-50%, such as 25-30%. In examples thereof more or less symmetrical configurations may be chosen.

In an example (see FIG. 3a) a top electrode layer 25 is provide on a piezoelectric layer 35 and further on a bottom electrode layer, the top electrode layer covering the piezoelectric layer at a left and right side thereof for approximately ⅔. The MEMS is attached to surrounding (silicon) material 15 at a left and right side thereof. In a less preferred alternative the MEMS is fully attached (i.e. also on the top and bottom thereof) to the surrounding material. More preferred is to attach the MEMS, if a rectangular structure is chosen, in its four corners to the surrounding material. If a polygon is chosen all corners may be attached, or a subset thereof, preferably a symmetrical (with respect to a center point of the MEMS) subset thereof. Such is relevant in view of energy efficiency (input power versus output ultrasound power).

In an example (not shown) a central section of the piezoelectric material may be absent, the central section forming 5-50% of the MEMS area (width*height). In a further alternative at least one corner section (typically all corner sections) of the piezoelectric material may be absent, the at least one corner section forming 5-60% of the MEMS area (width*height); in this latter case a remaining central section is attached to surrounding material by at least two relatively small bridges for electrical connection (e.g. 5-25 μm width).

In a further example (FIG. 3b) the MEMS may be pro-vided with at least one slit, typically one slit per 100-500 μm of MEMS (in a height or width direction), such as one per 200-250 μm, each slit having a width of 5-25 μm, such as 10-20 μm.

In FIG. 3c a configuration with corner attachments, and an absent central section of the piezoelectric material is shown.

In an exemplary embodiment the present MEMS has specific dimensions, wherein cross-sectional dimension, such as a width (or equivalently a length or diameter for a circular MEMS) [μm] of the MEMS is 400 (±40%)/ultrasound frequency [MHz], the ultrasound frequency being 0.1 MHz-60 MHz; in other words the dimension of the MEMS (expressed in μm) is 400 divided by ultrasound frequency (expressed in MHz); for example for 0.1 MHz the dimension is 4000 μm, for 1 MHz the dimension is 400 μm, and for 10 MHz the dimension is 40 μm. Due to the large range of frequencies there is some inherent uncertainty in the optimal ratio of 40%. The 40% also indicates that sub-optimal results may still be achieved if deviating from the exact ratio of 400. For better results however a smaller range of ±25% is preferred, whereas a range of ±10% is even more preferred, such as ±5%. It is further preferred that a length and width of the MEMS are almost or completely the same (±10% relative), that is being square. An alternative MEMS may be circular or polygonal, preferably hexagonal. It has been found that in view of power consumption and energy efficiency (e.g. in terms of power provided versus output ultrasound power) such configurations perform optimal (e.g. >80% efficiency (input/output) and typically >95% efficiency. Also further modifications, as detailed throughout the description, are considered in this respect.

For providing the MEMS with sufficient stiffness an adequate layer may be provided. This layer may be located at a bottom of the stack, at the top of the stack, in the middle of the stack, somewhere in the stack, and combinations thereof. In view of e.g. reliability, control, and robustness, such a layer is preferred.

With the above a reliable, durable and controllable MEMS is provided, which overcomes at least one of the prior art problems.

The present MEMS can be tailored, e.g. such that desired frequencies and/or powers can be obtained.

With the present MEMS and/or with comparable MEMS a portable ultrasound device can be constructed, which is suited for measuring liquids. In a preferred example the device is so small that it can be worn on e.g. a human body.

Also the present MEMS can be operated in such a fashion that less energy is consumed, a better reliability and durability is obtained, and accurate measurements can be obtained.

The present piezoelectric layers have a built-in poling in the vertical growth direction which is the same for all layers in the layer stack(s). The fact that the present layer stack may be grown inherently with the same poling direction has an advantage of a simplified processing, without need for bonding of separate elements, and of a built-in internal poling field at growth temperature of about 500° C., which is much more robust than poling by an external field after crystal growth. Also, in the pre-sent invention the piezoelectric (PZT) layers each have their own bottom and top electrode, and have an insulating layer between them, such that an electrical potential at all electrodes is independent from one and another. Thus with this different construction it is possible to have a field on each piezoeletric layer in a same vertical direction, which is found to act as a transducer for excitation of ultrasound waves with a larger amplitude.

The present invention provides a portable bladder monitor which can be activated and read out at any time or warns the person or caretakers if a certain threshold of urine volume is exceeded in the bladder. Also, for caretakers of bedridden and dementing patients who depend on help of other persons for a timely visit to the toilet, it is a great help to monitor the urine content in the bladder. For this a wireless readout of the bladder volume facilitates the caretaker to monitor if the patient needs help or not, independently of the patient. A wireless readout is there-fore a further option, next to a wired readout.

The present device can also be used for monitoring a condition of a patient during surgical operations in a hospital by sticking it on the skin of the patient, such as for monitoring a blood flow, blood pressure, need for catheterization, changing a diaper or providing a diaper, and heartbeat.

Thereby the present invention provides a solution to one or more of the above mentioned problems.

Further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIGS. 1a-1d illustrate MEMS built up according to the invention;

FIGS. 2a-m illustrate an exemplary process flow of the present invention; and

FIGS. 3a-c show examples of present MEMS designs.

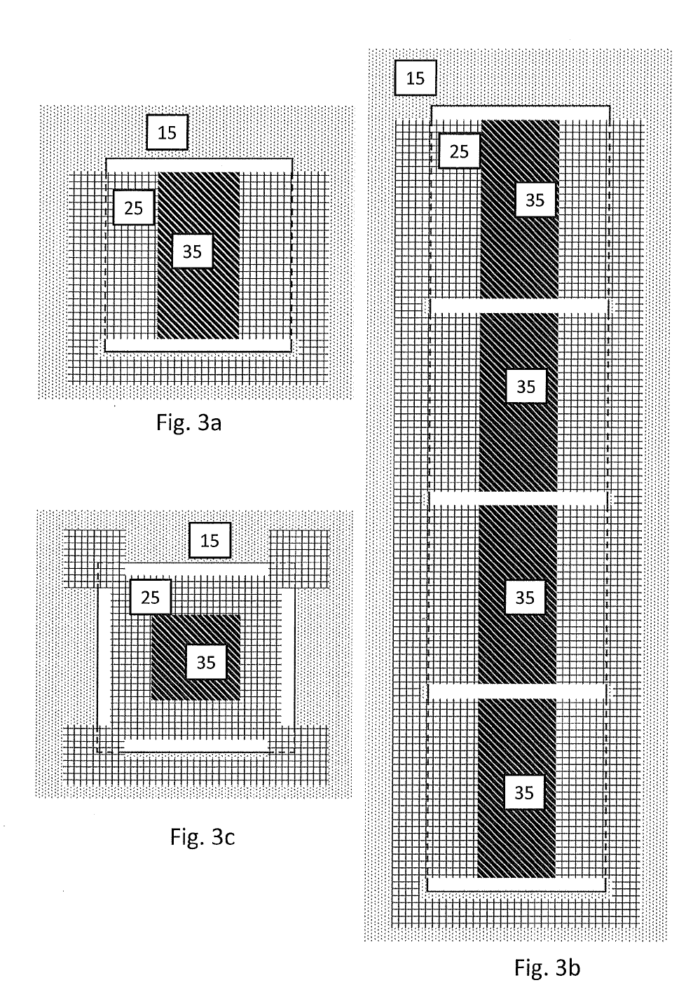

### DETAILED DESCRIPTION OF THE INVENTION

The present invention relates in a first aspect to a high voltage MEMS according to claim 13.

Microelectromechanical systems (MEMS) relates to a technology of very small devices; it comprises nanoscale nanoelectromechanical systems (NEMS) and nanotechnology. MEMS are also referred to as micromachines, or micro systems technology. Typically MEMS are made up of components between 1 to 5000 micrometers in size (i.e. 0.001 to 5 mm), and MEMS devices generally range in size from 20 micrometers to a millimeter. They usually consist of a central unit that processes data (the microprocessor) and several components that interact with the surroundings such as microsensors.

Piezoelectricity relates at one hand to accumulation of electric charge in certain solid materials in response to an applied mechanical stress. The word piezoelectricity means electricity resulting from pressure. The piezoelectric effect in optima forma may relate to linear electromechanical interaction between the mechanical stress and an electrical state (charge; i.e. electrical field) in e.g. crystalline materials. The piezoelectric effect is in principle a reversible process, however sometimes piezoelectric elements may malfunction, such as due to breakage; materials exhibiting a direct piezoelectric effect (internal generation of electrical charge resulting from an applied mechanical force) also exhibit a reverse piezoelectric effect (an internal generation of a mechanical strain resulting from an applied electrical field). For example, certain materials will generate measurable piezoelectricity when their static structure is deformed by about 0.1% of its original dimension. Conversely, those same crystals will change about 0.1% of their static dimension when an external electric field is applied to the material. The inverse piezoelectric effect is used in production of ultrasonic sound waves.

In an example of the present MEMS a configuration of the stack is symmetric. For instance a stack of two MEMS elements is provided, having an A-A form (two identical elements). In a further example an A-A-A stack, and an A-B-A stack is provided (a middle element being the same and different from both the outer elements, respectively). Such a symmetric stack provides a narrower bandwidth, better control, less wear, less power consumption, etc.

In an example the present MEMS comprises at least one of a MEMS cantilever, a MEMS double clamped beam, and a MEMS membrane.

In an example of the present MEMS a length of the MEMS is 10-2500 μm, preferably 15-1000 μm, more preferably 25-500 μm, such as 50-200 μm, and combinations thereof.

In an example of the present MEMS a width of the MEMS is 5-1000 μm, preferably 10-250 μm, more preferably 20-100 μm, and combinations thereof.

In an example of the present MEMS a thickness of the piezoelectric layer is 0.1-10 μm, preferably 0.25-5 μm, more preferably 0.5-2.5 μm.

In an example of the present MEMS a thickness of the electrode layer is 0.1-10 μm, preferably 0.25-5 μm, more preferably 0.5-2.5 μm.

In an example of the present MEMS a thickness of the dielectric layer is 0.1-10 μm, preferably 0.25-5 μm, more preferably 0.5-2.5 μm.

In an example of the present MEMS a thickness of the bottom layer is 1-500 μm, preferably 2.5-250 μm, more preferably 5-100 μm.

Some example of MEMS transducers for ultrasound wave excitation and sensing can be made smaller than half of a wavelength of the ultrasound waves. Such as for 6 MHz the wavelength could be 300 μm, and for 12 MHz it could be 150 μm in a medium with the density of water. At present ultrasound transducers are typically larger than 135 μm, which means that for wavelengths shorter than about 270 μm side lobes occur during beam steering. In order to improve e.g. ultrasound echography image, apodization filters in the software are provided to eliminate the side lobes in the image. If a pitch of the MEMS devices in the array is made less than half the wavelength, no side lobes occur and no apodization filters are necessary. Improved images without side lobes saves processing power for the image, it speeds up imaging which one would like to occur real time (which is not yet achievable for high resolution ultrasound imaging), and it improves the quality of the image. The above dimensions can be processed and integrated in typical semiconductor processes without much difficulty.

In an example of the present MEMS the electrode layer is selected from metals, such as Pt, Au, Cu, Al, W, Mo, TiN, Ti, a metallic conductor, and combinations thereof, preferably Pt.

In an example of the present MEMS the piezoelectric layer is selected from PZT (Pb[ZrₓTi₁₋ₓ]O₃ 0≦x≦1), AlN, (PbMg_{0.33}Nb_{0.67})₁₋ₓ(PbTiO₃)ₓ (preferably x=0.28-0.5) (PMNT), and combinations thereof, preferably PZT.

In an example of the present MEMS the dielectric layer is selected from SiO₂, Si₄N₃, and combinations thereof.

In an example of the present MEMS the bottom layer is selected from SiO₂, Si, SiC, Si₄N₃, and combinations thereof, preferably Si and Si₄N₃. The bottom layer is preferably Si or Si₄N₃, in view of producing the present MEMS, which provides good maintenance of characteristics of the present MEMS during producing.

Within a group of suitable materials, also combinations are envisaged.

In an example of the present MEMS an adhesive layer is present between an electrode layer and a piezoelectric layer.

In an example the present MEMS comprises a cavity or an ultrasound absorbing or quarter lambda reflecting (multi)layer. The cavity may be filled, such as with epoxy, or may be open (typically vacuum or filled with air). A difficulty with prior art MEMS cantilevers and many other transducers is that they generate a forward ultrasound wave which should be transmitted into the external medium under investigation, but it also sends out a wave from the back of the device into the cavity. The present absorber material, such as epoxy with an optimized thickness, absorbs this backward travelling wave. For prior art MEMS, which are not open on the backside, filling up the cavity below the MEMS cantilever is difficult if not impossible as there is no opening accessing the cavity.

In an example the present MEMS comprises 2-220 piezoelectric elements, preferably 3-210 piezoelectric elements, more preferably 4-25 piezoelectric elements. High end applications, such as for 3D-imaging, may have a large number of piezoelectric elements, such as 214. Application such as a bladder monitor may have a relative small number of elements. Medium end applications, wherein for instance some image formation is required, may have 10-1000 piezoelectric elements. To each piezoelectric element 20-200 V may be applied, such as 50 V. The voltage may be applied by one voltage source, and splitting the voltage to the piezoelectric elements.

In an example of the present MEMS the piezoelectric layer is a laser assisted sputtering layer. It has been found that such piezoelectric layers have as characteristics that the layer does not blister, has an intrinsic electrical polarity, is stable and reliable, etc., contrary to most prior art piezoelectric layers, and in particular PZT-layers.

The above present layer is in an example an in a perpendicular growth direction monocrystalline layer. In a further example it may be characterized by crystalline granular elements and/or bubbles.

In an example of the present MEMS at least one piezoelectric layer has an intrinsic electrical polarity, preferably all piezoelectric layer have an intrinsic electrical polarity, wherein the polarity is larger than 20 V/μm, preferably larger than 50 V/μm, more preferably larger than 100 V/μm, such as 200-1000 V/μm. The present polarity is typically parallel to a growth direction. Therewith for instance linear behavior between applied electrical field and change in dimension(s) of the piezoelectric layer is provided, contrary to prior art layers without or with at the most a limited intrinsic polarity. The intrinsic polarity is preferably at least as large as an external electrical field to be applied, more preferably significantly larger, in view of the above.

In a second aspect the present invention relates to a portable ultrasound device. The present device comprises at least one ultrasound transducer, the transducer comprising at least one MEMS, the MEMS comprising at least one piezoelectric element, and a cavity or an ultrasound absorbing or reflecting (multi)layer. Therewith ultrasound can be provided at a sufficient intensity.

The portable device is a small device, to be carried by a single person, to be applied e.g. to a person, etc.

The portable device comprises a voltage source for applying a voltage to the transducer, preferably a high voltage source, such as a source providing 20-500 V, preferably 50-250 V, such as 80 V. Likewise the present MEMS could comprise such a source. The source may also be considered as an actuator.

In an example the portable device comprises a voltage splitter, for applying a voltage to an individual piezoelectric element.

The portable device comprises a means for providing electrical energy, such as an electrical energy source, and an energy converter. Examples of an electrical energy source are a battery, and a capacitor. Likewise an energy converter may be used, such as a converter that converts body warmth into electricity, movement into electricity, pressure into electricity, etc.

The portable device comprises a detector for detecting reflected ultrasound. The detector and MEMS of the device are preferably one and the same.

The present portable device can comprise an integrated scanner system of a transducer (set of transducers, an integrated series of transducers, a (series of) MEMS transducers, piezoelectric transducers integrated with the high voltage actuation circuit and/or sensing circuit and/or data processing and a battery power supply in the same package. In an example this monitoring scanner (or likewise scan head) may connect to a read out system, wired or wireless connected, for measuring and calculation of the bladder/urine volume, and communication of this value like by a display or alarm function.

The present scanner may be thin and can be worn by a person in or under a dress or underwear, mounted by an adhesive on the skin, or fixed on the skin, such as with a strap.

In an example the transducer in the scanner can generate ultrasound pulses in a range of about 1 MHz to 10 MHz, and can also detect ultrasound echoes, such as from a front and a back of the bladder. Then, from a measurement of difference in time lapse between transmission of the actuation of a pulse (or signal) and the reception of the above two echoes a volume of the liquid in the bladder can be calculated. This volume is considered a measure of the volume of urine in the bladder.

If this volume of urine exceeds a certain value, a pulse provided by a transmitter, such as an alarm, like a beep or vibration, may warn a person, such as to visit to the toilet/urinal. The transmitter may be located in the present scanner or likewise device, or outside thereof, or a combination thereof.

An actuation of a pulse transmitter can be automatic periodically or it can be activated manually by the person.

The present supporting electronics may include at least a battery management circuit enabling several days or weeks or longer of battery life by management the stand by function with low power consumption, a high voltage circuit for the transmit pulse on the piezoelectric transducers, a receive/sense circuit for detection of the echo and time between the echo's, and possibly in the same package, data processing and communication circuit, display circuit or wireless RF, or wired transmission.

For ultrasound generation and detection, respectively, transducers can be used, such as piezoelectric devices, PZT MEMS, single crystalline MEMS, and capacitive MEMS.

A few applications of the present portable ultrasound device are given below.

A device for personal use as a continuous monitor is envisaged. It can be read at any moment in order to observe the volume of the urine bladder, constructed of transducer(s) for transmitting ultrasound vibrations (around 1-10 MHz), sensing the echo of these vibrations, electronics for generating the transmit pulse (can be tens to hundreds of volts), and sensing circuit, processing in hardware and software for the interpretation of the echo's as a volume of the urine bladder. This device is small enough to be wearable and can be fixed by a piece of tape or adhesive plaster or elastoplast.

It can relate to a device with two or more transducer elements (MEMS or non-MEMS) placed in a same package, and to one package all together. Such is a thin device for portable purpose, such as mounted by a fixator, such as a strap, or stuck with a glue, or fixed in the (under)wear. This may be connected, wired or wireless, with an electronic readout with a display which may also be portable on the body, possibly in a trouser pocket or under the belt, and/or which is an app on the mobile phone-like device.

The present device may have a compact PCB or chip with a high voltage driver and/or integrated or separate sensor readout circuit in a single package, and further comprising a battery with a battery management circuit. Therewith a bundle of high voltage cables between transducers in the scanner and high voltage circuits for driving the transducers is replaced. A problem which is solved is that a thick bundle of high voltage cables is stiff and may cause a serious strain on the user who moves the ultrasound scanner manually. Especially for frequent users such as medical assistants and doctors this may cause RSI. By omitting the high voltage cable, only a voltage supply wire and light digital wiring is needed, in a much lighter and more flexible cable. This results in a higher convenience for the user. Another problem is that the driving power of the high voltage drive circuit is dimensioned on driving the charge for the cable mainly. If the cable is omitted, far less driving power is needed, and the circuits on the chip can become much smaller, which facilitates a small footprint of the high voltage chip. The power savings are larger if the operating frequency of the ultrasound scanner is higher, for instance more than 10 MHz.

The present package also saves considerable electrical driving power, which offers the advantage of less heating of the high voltage driver chip. For a handheld device the power consumption could be limited to about 4 Watt, in order to prevent inconvenient heating of the handheld scan head.

The present device may be (in combination with) an APP on a mobile phone-like (or iPad) device for wireless readout, in order to display the calculated volume of the bladder and urine.

The present device may have an alarm function, for providing an alarm in case the calculated volume exceeds a certain threshold limit.

The present device may be for continuous or semi-continuous monitoring of ballooning of arteries, aorta, and blood vanes, possibly located close to the bladder. With a higher resolution than needed to detect the volume of the bladder, for instance by using MEMS transducers, an image of blood vanes is possible which results in an image. Using an image it can be observed if ballooning occurs. Bursting blood vessels can lead to death by internal bleeding if no medical surgery is applied within hours.

In an example the present portable device comprises a MEMS according to the invention.

The portable device provides ultrasound signals. Depending on e.g. the MEMS the signals are in a range of about 20 kHz to about 50 MHz. Also combinations of frequencies are envisaged.

In an example the present portable device comprises 2-220 transducers, preferably 3-100 transducers, such as 4-6 transducers. Therewith a large variation in power(s) and/or frequencies can be provided. Also a series of transducers provides an ultrasound (combined) signal, which signal provides more accurate information, e.g. on an amount of liquid. This allows e.g. for broad resonance mode actuation, build from the adjacent resonant frequencies. It requires less damping for a broad frequency spectrum compared to prior art systems as the broadening per peak can be less if several peaks of adjacent frequencies are excited simultaneously. This allows for a better energy efficiency and it saves power in the scan head, which will heat up less.

In an example of the present portable device the voltage source and the at least one transducer are in direct contact. Therewith power consumption, signal distortion, reliability etc. are improved. In an example a low capacitance contact is provided, such as by a bond wire, bond ball, and interconnect.

In an example the present portable device consists of one integrated package. Dimensions thereof are typically 1-10 mm by 1-10 mm, and a thickness of 0.1-2 mm. If the present package is integrated in a portable device dimensions may be 1-0 cm by 5-20 cm and a thickness of 0.2-5 cm.

In an example the present portable device comprises a transceiver, preferably a wireless transceiver, such as an RFID, for communicating with an outside world.

In an example the present portable device comprises a unique identification code. The code identifies the present device and/or a user thereof. As such it can be directly clear which device, e.g. allocated to a person, provides e.g. a measurement. Thereafter, if required, appropriate measures can be taken.

In an example the present portable device comprises at least one threshold, the threshold for determining a pre-set unique amount of liquid. Therewith for any individual device and/or any individual user thereof a threshold may be provided, e.g. for determining a minimum value to be measured, the minimal value giving a motivation to act, e.g. to change a diaper or to urinate.

In an example the present portable device comprises at least one apodization filter. The filter may correct for signals provided by the present system and reflections obtained.

In an example of the present portable device the cavity comprises an ultrasound absorbing material, such as epoxy. Therewith unwanted ultrasound signal are substantially blocked.

In an example of the present portable device the device is one or more of disposable, such as a blister, a handheld device, such as a scanner, . . . . The present device may be relatively small, essentially comprising an integrated package, to be applied “directly”, and may be somewhat larger, e.g. in the form of a scanner or a warning device. If a scanner or warning device is provided it is preferred to combine the present device with an image forming technique; thereby a user can inspect the image directly, e.g. in view of location of liquid, location of an obstacle, etc.

In an example the present portable device comprises a series of MEMS, each MEMS individually providing an ultrasound having a frequency and a power, the series providing a multi-frequency spectrum of ultrasounds and/or powers. Therewith an adaptable signal can be provided, for obtaining reliable and adequate results. In an example at least two MEMS have a cavity or an ultrasound absorbing or reflecting (multi)layer in common, optionally providing coherent ultrasound. Such is an important advantage of the present method of producing the present MEMS, which method is detailed in the examples.

In an example the present portable device is for detecting a liquid volume, such as in a body part, such as in a bladder, in a joint, and in a blood vessel, for ultrasound image forming, such as in an endoscope, for warning, such in a car-parking system. The present MEMS allows for more elements to be placed in a very limited (less than typically 0.5 cm) space of an endoscope. With the present MEMS more elements can be put into the endoscope allowing for higher resolution imaging of the tissue surrounding the endoscope.

In a third aspect the present invention relates to a method of operating an ultrasound device according to the invention, comprising the steps of determining an amount of liquid in a bladder, based on the amount deter-mined, taking a further action, such as going to a toilet, and changing a diaper. As such a person wearing a diaper, a (professional) health care provider, etc. can be signaled to change the diaper, e.g. because the person wearing the diaper is in need of a visit to the toilet. Likewise the person can go to the toilet, or being assisted therein.

In an example of the present method the ultrasound device provides a signal if a pre-set unique amount of liquid is exceeded, such as by a sound, an optical signal, vibration, wireless communication to an observer, to a smartphone, to a mobile phone, to an app, to a computer, to a server, wherein the signal preferably comprises a unique code identifying a person, and a location of said person. Thereafter appropriate action can be taken.

In a fourth aspect the present invention relates to a method of operating a MEMS according to the invention, wherein a first voltage is applied to a first piezoelectric layer, and a second voltage is applied to a second piezoelectric layer, wherein the first and second piezoelectric layer are optionally symmetrical layers, and wherein the first voltage provides a shrinkage to the first layer and the second voltage provides an elongation to the second layer, wherein the shrinkage and elongation are adapted to one and another. Therewith e.g. durability, reliability, power consumption, and quality of use of the present MEMS are improved.

In a further method, to be combined with the above, or to be carried out separately, the present MEMS is operated by applying a bias voltage for compensating internal stress. As such the quality of the present MEMS is improved.

In a fifth aspect the present invention relates to a membrane for use in an ultrasound device according the invention, comprising a membrane providing stiffness, at least two MEMS according to the invention, preferably comprising at least one series of MEMS, each MEMS individually providing an ultrasound having a frequency and a power, the series providing a multi-frequency spectrum of ultrasounds and/or powers, wherein preferably at least two MEMS have a cavity or an ultrasound absorbing (multi)layer in common, optionally providing coherent ultrasound, preferably 2-50 series of MEMS, wherein the bottom layer for providing stiffness of the MEMS and the membrane are optionally one and the same. Therewith a membrane is provided that can be incorporated into a further device for ultrasound.

The invention is further detailed by the accompanying figures and examples, which are exemplary and explanatory of nature and are not limiting the scope of the invention. To the person skilled in the art it may be clear that many variants and combinations of the exemplary embodiments, being obvious or not, may be conceivable falling within the scope of protection, defined by the present claims.

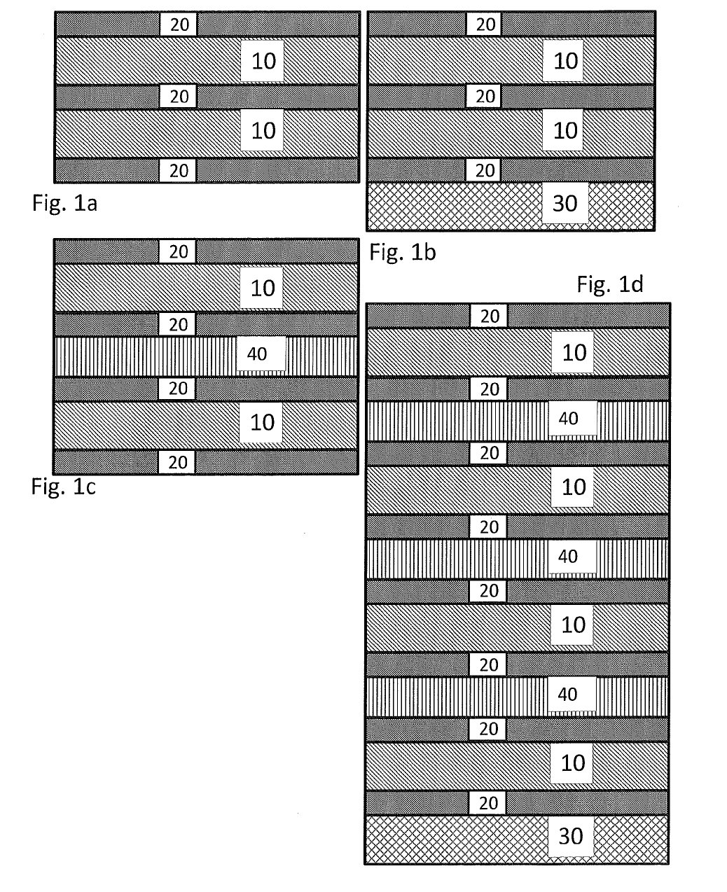

In FIG. 1a a basic piezoelectric element is shown. Therein, from top to bottom, a top electrode layer 20, a piezoelectric layer 10, a bottom electrode 20, also functioning as a top electrode layer 20, a piezoelectric layer 10, and a bottom electrode 20 are shown. To the top electrode a first voltage may be applied, to the middle electrode a second potential, and to the bottom electrode a third potential, such as +50 V, 0 V, −50 V, and 100 V, 50 V and OV, respectively. A voltage may be provided as such, or as a split voltage from one source.

In FIG. 1b, in addition to FIG. 1a, a stiff layer 30 is present, such as a SiN layer. The layer may be at the bottom, it may be at the top, and both. Further a stiff layer may be present in between the bottom electrode 20 of the top piezoelectric layer, and the top electrode 20 of the bottom electrode layer, in which case the bottom and top electrode are not the same.

In FIG. 1c, compared to FIG. 1a, a dielectric layer 40 in between the bottom electrode 20 of the top piezoelectric layer, and the top electrode 20 of the bottom electrode layer, is present.

In FIG. 1d four piezoelectric elements, each comprising a top electrode layer 20, a piezoelectric layer 10, and a bottom electrode layer 20, with in between a dielectric layer 40, and a stiff layer 30 is present. Each piezo-electric layer may have a voltage of e.g. 50 V, which may be a split voltage from one single source. A total voltage over the layers would then be 200 V.

Further details of the figures are given throughout the description.

### EXAMPLES

The invention although described in detailed explanatory context may be best understood in conjunction with the accompanying examples and figures.

Process steps for making a MEMS (some steps are left out, as well as indication to some layers) include:

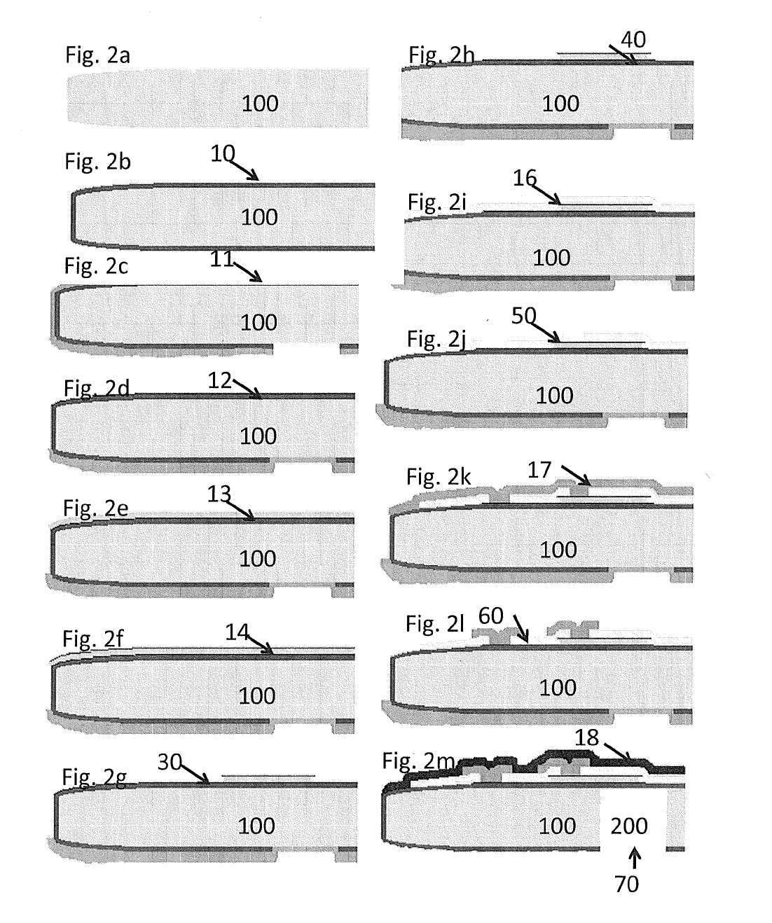

### Batch Formation (FIG. 2a)

get starting material (100; Si)
set up track and trace
laser marking
prepare for transport

### Double Side Markers on Wafer

thermal pad oxidation
(double side) markers
oxide etch

### Nitride Deposition (FIG. 2b)

LPCVD Nitride (10) deposition
   Backside Cavity Definition (FIG. 2c) (Having polySi 11)
SiN etch

### Bottom Electrode Deposition (FIG. 2d)

Pt electrode (12) deposition

### Piezo Layer Deposition (FIG. 2e)

PZT (13) deposition

### Top Electrode Deposition (FIG. 2f)

Pt (14) deposition

### Piezo Layer Definition (FIG. 2g)

Pt etch (30)
piezo layer etch

### Bottom Electrode Definition (FIG. 2h)

Pt etch (40)

### Separation Layer Deposition (FIG. 2i)

PECVD SiO₂ (16)

### Contact Hole Definition (FIG. 2j)

SiO₂ etch (50)

### Contact Deposition (FIG. 2k)

Al (17) deposition

### Interconnect Definition (FIG. 2l)

Al etch (60)

### Top Layer Scratch Protection Deposition (FIG. 2m)

PECVD SiN (18)

### Backside Cavity (200) Formation

KOH etch (70)

### Nitride Opening

SiN etch

### Nitride Release

SiN etch

### Packaging

What is claimed is:

1. A portable ultrasound device comprising:
at least one ultrasound transducer, the transducer comprising at least one MEMS, the MEMS comprising at least one piezoelectric element, a cavity, and one or more of an ultrasound absorbing layer, and an ultrasound reflecting layer,
a voltage source for applying a voltage to the transducer,
a means for providing electrical energy, and
a detector for detecting reflected ultrasound,
wherein the MEMS comprises a stack of layers, the stack comprising at least two piezoelectric elements poled in a same direction, each piezoelectric element comprising:
a top electrode layer,
a piezoelectric layer, and
a bottom electrode layer, wherein the top electrode covers the piezoelectric layer completely or partially, and wherein the piezoelectric layer covers the bottom electrode completely or partially.

2. The ultrasound device according to claim 1, additionally comprising a voltage splitter for applying a voltage to an individual piezoelectric element.

3. The ultrasound device according to claim 1, comprising 2-20 transducers.

4. The ultrasound device according to claim 1, additionally comprising at least one of a transceiver, a unique identification code, at least one threshold, the threshold for determining a pre-set unique amount of liquid, and at least one apodization filter.

5. The ultrasound device according to claim 1, wherein the at least one cavity comprises an ultrasound absorbing material, the voltage source and the at least one transducer in direct contact, and the portable device consists of one integrated package.

6. The ultrasound device according to claim 1, wherein the device is at least one of a disposable and a handheld device.

7. The ultrasound device according to claim 1, comprising a series of MEMS, each MEMS individually providing an ultrasound having a frequency and a power, the series providing a multi-frequency spectrum of ultrasounds and/or powers.

8. The ultrasound device according to claim 1, for one or more of the group consisting of measuring a liquid volume, ultra-sound image forming, and warning.

9. A method of operating an ultrasound device according to claim 1, comprising the steps of:
determining an amount of liquid in a bladder, and
based on the amount determined, taking a further action.

10. A high voltage MEMS for use in an ultrasound device comprising a stack of layers, the stack comprising at least two piezoelectric elements poled in a same direction, each piezoelectric element comprising:
a top electrode layer,
a piezoelectric layer, and
a bottom electrode layer, wherein the top electrode covers the piezoelectric layer completely or partially, and wherein the piezoelectric layer covers the bottom electrode completely or partially.

11. A MEMS according to claim 10, wherein a cross-sectional dimension in μm of the MEMS is 400 (±40%) and the MEMS provides an ultrasound frequency of 0.1 MHz-60 MHz.

12. The MEMS according to claim 10, comprising at least one of:
at least one dielectric layer in between two piezoelectric elements, and
a layer for providing stiffness to the stack of layers
wherein a configuration of the stack is symmetric,
a slit,
a connecting bridge, and
a MEMS cantilever, a MEMS double clamped beam, and a MEMS membrane.

13. The MEMS according to claim 10, wherein at least one of the following is provided:
a length of the MEMS is 10-2500 μm,
a width of the MEMS is 5-1000 μm,
a thickness of the piezoelectric layer is 0.1-10 μm,
the electrode layer is selected from metals, and metallic conductors,
the piezoelectric layer is selected from PZT, AlN, PMNT, and combinations thereof,
the dielectric layer is selected from SiO₂, and Si₄N₃,
the bottom layer is selected from SiO₂, Si, SiC, and Si₄N₃, an adhesive layer is present between an electrode layer and a piezoelectric layer,
comprising a cavity, and at least one of an ultrasound absorbing (multi)layer, and ultrasound reflecting (multi)layer,
2-220 piezoelectric elements, and
the piezoelectric layer is a laser assisted sputtering layer,
wherein the piezoelectric layer comprises crystalline granular elements, and
at least one piezoelectric layer has an intrinsic electrical polarity.

14. A method of operating a MEMS according to claim 10, wherein a first voltage is applied to a first piezoelectric layer, and a second voltage is applied to a second piezoelectric layer, and wherein the first voltage provides a shrinkage to the first layer and the second voltage provides an elongation to the second layer, wherein the shrinkage and elongation are adapted to one and another, and/or applying a bias voltage for compensating internal stress.

15. A membrane for use in an ultrasound device comprising:
a membrane providing stiffness, and
at least two MEMS according to claim 10, each MEMS individually providing an ultrasound wave having a frequency and a power, the series providing a multi-frequency spectrum of ultrasounds and/or powers.
